(19) United States
(12) Patent Application Publication
(10) Pub. No.: US 2013/0123786 A1
(43) Pub. Date: May 16, 2013

(54) PEDICLE SUBTRACTION OSTEOTOMY DEVICE AND METHODS

(71) Applicant: Bruce M. McCormack, San Francisco, CA (US)

(72) Inventor: Bruce M. McCormack, San Francisco, CA (US)

(21) Appl. No.: 13/670,245

(22) Filed: Nov. 6, 2012

Related U.S. Application Data
(60) Provisional application No. 61/560,674, filed on Nov. 16, 2011.

(51) Int. Cl. A61B 17/16

(52) U.S. Cl. CPC A61B 17/1671
USPC 606/82

(57) ABSTRACT

Disclosed herein is a device for cutting bone in performing a pedicle subtraction osteotomy. The device may include an elongated member and a flexible cutting member. The elongated member includes a distal end, a proximal end, an elongated slot between the distal end and proximal end and extending generally parallel to a longitudinal axis of the elongated member, and a fulcrum located distal the proximal end. The elongated member is configured to be anchored to the bone. The flexible cutting member extends through the elongated slot and around the fulcrum such that at least a portion of the flexible cutting member can extend from the elongated slot to cut the bone as the flexible cutting member is moved back and forth in a sawing fashion about the fulcrum. The elongated member may be in the form of a threaded bone screw. The flexible cutting member may be in the form of an abrasive or tooth equipped wire, cable or rope.

### CROSS-REFERENCE TO RELATED APPLICATION

The present application incorporates in its entirety and claims the benefit under 35 U.S.C. §119(e) of: U.S. Provisional Application 61/560,674 filed Nov. 16, 2011 and titled Pedicle Subtraction Osteotomy Device and Methods.

### FIELD OF THE DISCLOSURE

Aspects of the present disclosure relate to medical apparatus and methods. More specifically, the present disclosure relates to devices and methods for performing a pedicle subtraction osteotomy.

### BACKGROUND OF THE DISCLOSURE

A fixed sagittal imbalance or flat back syndrome is a spinal deformity that causes the center of gravity, which typically falls through the sacral promontory in the standing position when the spine is balanced and healthy, to be located in front of the sacrum. Patients with this syndrome cannot stand erect without flexing their knees and hips, and experience pain when standing due to fatigue of the spinal extensor muscles which are mechanically disadvantaged. The syndrome commonly occurs in middle aged patients with scoliosis treated with Harrington instrumentation extending to the mid lumbar spine. Hardware distracts out normal spinal curvatures and pitches the spine forward. With aging, the lower lumbar segments degenerate and lose their ability to hyperextend to compensate for the cephalad kyphosis. There is a progressive loss of sagittal balance with aging. The syndrome also occurs after instrumentation of thoracolumar fractures, lumbar arthrodesis, and multilevel laminectomy. The syndrome occurs in patients with ankylosing spondylitis.

The two most commonly used procedures to treat sagittal imbalance include the Smith Petersen osteotomy and pedicle subtraction osteotomy. The pedicle subtraction osteotomy is more favored of the two procedures as it provides an additional advantage of obtaining correction through all three spinal columns with a posterior approach, and maximizes the healing potential. In its current form, however, osteotomy is a technically difficult procedure that is typically done by highly trained surgeons at tertiary spine centers.

Current techniques for osteotomy entail the use of drills, osteotomes and other hand tools that make multiple imprecise bony cuts. This inexact cutting causes unreliable correction of deformity and coronal imbalance if, for example, one side of the vertebra is cut more deeply than the other. Also, surgeries are usually long and bone bleeding can be significant. Nerve injury is another complication that can occur due to the lack of standardized technique. Thus, because of these aforementioned challenges associated with this otherwise successful surgical technique, its widespread adoption has been suppressed and flat back syndrome remains under treated.

Accordingly, there is need in the art for a device for, and method of, safely and reliably performing a pedicle subtraction osteotomy.

### BRIEF SUMMARY OF THE DISCLOSURE

Disclosed herein is a device for cutting bone in performing a pedicle subtraction osteotomy. In one embodiment, the device includes an elongated member and a flexible cutting member. The elongated member includes a distal end, a proximal end, an elongated slot, and a fulcrum. The elongated slot is between the distal end and proximal end and extends proximal-distal. The fulcrum is located distal the proximal end. The elongated member configured to be anchored to the bone. The flexible cutting member extends through the elongated slot and around the fulcrum such that at least a portion of the flexible cutting member can extend from the elongated slot to cut the bone as the flexible cutting member is moved back and forth in a sawing fashion about the fulcrum.

In one version of the device, the elongated member includes a threaded bone screw, and the flexible cutting member includes an abrasive or tooth equipped wire, cable or rope.

In one version of the device, the elongated slot in extending proximal-distal extends generally parallel to a longitudinal axis of the elongated member. In one version of the device, the proximal end includes a head configured for screwdriver or wrench engagement, and the elongated slot may extend through a side of the head.

In one version of the device, the elongated member further includes a channel extending along a longitudinal axis of the elongated member. The elongated slot opens into the channel. In addition to the flexible cutting member extending through the elongated slot and around the fulcrum, the flexible cutting member also extends through the channel to exit out the proximal end.

In one version of the device, the elongated member comprises a threaded outer surface. Further, the proximal end may be configured for screwdriver or wrench engagement and the distal end may be tapered.

In one version of the device, the fulcrum includes a plurality of fulcrums spaced apart from each other along the elongated member. Depending on the version of the device, the fulcrum may include a pulley, a pin, or a ledge defined in the slot. In one version of the device, the elongated member further includes a radiopaque marker indicating a location of the fulcrum.

In one version of the device, the slot may include a first slot and a second slot generally opposite the elongated member from the first slot. The first slot may extend distally further along the elongated member than the second slot.

In one version of the device, the slot includes a first fulcrum ledge and a second fulcrum ledge. The first fulcrum ledge is located more distal on the elongated member than the second fulcrum ledge. Also, the first fulcrum ledge may be located on generally an opposite side of the elongated member from the second fulcrum ledge.

In one version of the device, the slot further comprises a segment routed both distal-proximal along the elongated member and circumferentially about at least a portion of a circumference of the elongated member.

Also disclosed herein is another device for cutting bone in performing a pedicle subtraction osteotomy. In one embodiment, the device includes an elongated member and a flexible cutting member. The elongated member includes a fulcrum and a length distally terminating in a distal end. The elongated member is configured to anchor in bone when the length is imbedded in bone. The fulcrum is located proximal the distal end at a location of the elongated member that would be within bone when the length is imbedded in bone. The flexible cutting member is supported on the elongated member such that the flexible cutting member can be displaced back and forth about the fulcrum in a sawing fashion.

In one version of the device, the elongated member is configured such that a first portion of the flexible cutting member on a first side of the fulcrum extends through the elongated member to exit the elongated member at a proximal end of the elongated member. The elongated member is further configured such that a second portion of the flexible cutting member on a second side of the fulcrum opposite the first side of the fulcrum exits the elongated member through a slot extending longitudinally along a side of the elongated member. The slot extends proximal-distal generally parallel to a longitudinal axis of the elongated member.

In one version of the device, the proximal end comprises a head configured for screwdriver or wrench engagement. The slot may extend through a side of the head.

In one version of the device, the elongated member further includes a channel extending along a longitudinal axis of the elongated member. The slot opens into the channel and the first portion of the flexible cutting member extends through the channel in extending through the elongated member.

The fulcrum may be a pulley, a pin, or a ledge defined in the slot. In being configured to anchor in bone, the elongated member may include a threaded exterior configuration. Also, the flexible cutting member may include an abrasive or tooth equipped wire, cable or rope. A radiopaque marker on the elongated member may indicate a location of the fulcrum.

Also disclosed herein is a method of cutting bone in performing a pedicle subtraction osteotomy. In one embodiment, the method includes: anchoring a first elongated member to a first location on the bone such that a fulcrum supported on the first elongated member is located inside the bone; and moving a flexible cutting member back and forth about the fulcrum to cause the flexible cutting member to cut the bone.

In one version of the method, the fulcrum is supported on the first elongated member near a distal end of the first elongated member. The distal end of the first elongated member is embedded inside the bone.

In one version of the method, the method further includes causing at least a portion of the flexible cutting member to extend from an elongated slot defined in the first elongated member. Also, the anchoring may include screwing the first elongated member into the bone. More specifically, the anchoring may include screwing the first elongated member into the bone such that at least a distal end of the elongated member becomes embedded in the bone.

In one version of the method, the method further includes: anchoring a second elongated member to a second location on the bone such that a fulcrum supported on the second elongated member is located inside the bone; and causing the flexible member to extend across the fulcrum of the first elongated member and the fulcrum of the second elongated member. In such a version of the method, the step of moving the flexible cutting member back and forth about the fulcrum to cause the flexible cutting member to cut the bone further includes moving the flexible cutting member back and forth about the first and second fulcrums to cause the flexible cutting member to cut the bone.

In one version of the method, the first location on the bone may include a first vertebral pedicle subsequent to a laminectomy and facetectomy. Also, the second location on the bone may include a second vertebral pedicle subsequent to a laminectomy and facetectomy. The flexible cutting member may be caused to extend across a vertebra between the first and second locations and under a dura. Also, a shield may be located between the flexible cutting member and a nerve structure. Further, the shield may be located between the first and second locations.

In one version of the method, a first segment of the flexible cutting member is joined to a second segment of the flexible cutting member to form the flexible cutting member. The elongated member may include a threaded exterior configuration. Also, the flexible cutting member may include an abrasive or tooth equipped wire, cable or rope.

While multiple embodiments are disclosed, still other embodiments of the present disclosure will become apparent to those skilled in the art from the following detailed description, which shows and describes illustrative embodiments of the disclosure. As will be realized, the systems, devices and methods disclosed herein are capable of modifications in various aspects, all without departing from the spirit and scope of the present disclosure. Accordingly, the drawings and detailed description are to be regarded as illustrative in nature and not restrictive.

### BRIEF DESCRIPTION OF THE DRAWINGS

FIG. 1A is an isometric view of an embodiment of a device intended for use in the pedicle subtraction osteotomy, wherein the device is in the form of a screw assembly.

FIG. 1A-1 is an enlarged view of a distal region of the device of FIG. 1A.

FIG. 1A-2 is a side view of a fulcrum supported in a distal region of the device of FIG. 1A.

FIG. 1B is an isometric view of another version of the device.

FIG. 1B-1 is a longitudinal cross section of the device as taken along section line 1B-1-1B-1 in FIG. 1B.

FIG. 1B-2 is a transverse cross section of the device as taken along section line 1B-2-1B-2 in FIG. 1B.

FIG. 1C is an isometric view of yet another version of the device.

FIG. 1C-1 is the same device as depicted in FIG. 1C, except shown in an opposite isometric view.

FIG. 1C-2 is a longitudinal cross section of the device as taken along section line 1C-2-1C-2 in FIGS. 1C and 1C-1.

FIG. 1C-3 is a transverse cross section of the device as taken along section line 1C-3-1C-3 in FIGS. 1C and 1C-1.

FIG. 1D is an isometric view of yet another version of the device.

FIG. 1D-1 is the same device as depicted in FIG. 1D, except shown in an opposite isometric view.

FIG. 1D-2 is a longitudinal cross section of the device as taken along section line 1D-2-1D-2 in FIGS. 1D and 1D-1.

FIG. 1D-3 is a transverse cross section of the device as taken along section line 1D-3-1D-3 in FIGS. 1D, 1D-1 and 1D-2.

FIG. 1D-4 is a transverse cross section of the device as taken along section line 1D-4-1D-4 in FIGS. 1D-1 and 1D-2.

FIG. 1D-5 is a transverse cross section of the device as taken along section line 1D-5-1D-5 in FIGS. 1D and 1D-2.

FIG. 2 is a sagittal view of a deformed spine of a patient scheduled to undergo a pedicle subtraction osteotomy, wherein the center of gravity is illustrated as falling anterior of the sacrum.

FIGS. 3A, 3B and 3C are, respectively, sagittal, superior-sagittal isometric, and superior views of the selected spine segment, prior to the commencement of the procedure.

FIGS. 4A and 4B are, respectively, superior-posterior isometric and superior views of the vertebra following the laminectomy, facetectomy and removal of the pedicle.

FIG. 5 is the same view as FIG. 3A, except indicating a vertebial osteotomy site subsequent to the laminectomy and facetectomy.

FIG. 6 is the same view as FIG. 3B, except subsequent to the laminectomy and facetectomy and illustrating an embodiment of the device in the form of a screw assembly, such a screw assembly being inserted into the vertebral body through the each respective pedicle.

FIG. 7A is the same view as FIG. 6, except illustrating the pedicle and vertebral body resection process according to one embodiment of the current disclosure

FIG. 7B is a cross sectional view of the pedicle and vertebral body resection process as taken along section line 7B-7B in FIG. 7A.

FIG. 8 is the same view as FIG. 4A, except of a bone wedge or block formed at each pedicle after a simple circumferential pass of a single flexible cutting member extending from the respective screw assembly inserted in the respective pedicle.

FIG. 9 is the same view as FIG. 8, except showing the bone wedges and screw assemblies removed en block according to one embodiment of the current disclosure.

FIG. 10A is the same view as FIG. 8, except showing the bone wedges removed with each screw assembly still engaged with the vertebra body in accordance with another embodiment.

FIGS. 10B and 10C are each the same view as FIG. 7B, except of the osteotomy procedure performed according to one of the embodiments

FIGS. 11A and 11B are each the same view as FIG. 7B, except of the osteotomy procedure performed according to another embodiment.

FIGS. 12A-12C are, respectively, superior-sagittal isometric, superior-posterior isometric, and superior-posterior isometric views of the osteotomy procedure performed according to another embodiment.

FIGS. 12D-12F are each superior-posterior isometric views of the vertebra illustrating steps of employing pairs of cutting devices for laminectomy, facetectomy and removal of the pedicle.

FIG. 13 is a sagittal view of the spine illustrating one of the steps in the subtraction osteotomy process wherein four pedicle screws are inserted in the pedicles surrounding the osteotomy site.

FIG. 14 is the same view as FIG. 13, except of another of the steps in the subtraction osteotomy process wherein a wire is threaded through the heads of the pedicle screws in order to connect all of the screws together.

FIG. 15 is a sagittal view of the spine after the sagittal balance is restored, showing the center of gravity falling through the sacral promontory.

### DETAILED DESCRIPTION

A novel instrument and a method for performing a pedicle subtraction osteotomy are disclosed herein. A cutting device 10 is designed such that the complications that are typically associated with the use of currently available tools and procedures can be minimized, and thus allow for widespread use of an otherwise successful surgical technique and treatment.

As discussed in detail below, in one embodiment, the cutting device 10 includes a longitudinal screw assembly 15 and one or more flexible cutting member(s) 70 extending through a longitudinally directed groove or grooves 65 on the side of the screw shaft 35, or alternatively, within a hollow center of the screw shaft, or both. The longitudinal screw assembly 15 includes a fulcrum member 60 that serves as a fulcrum for a flexible bone cutting member(s). The fulcrum member is attached to, or defined in, the inner surface 55 of the screw shaft 35, and is located at or near the tip 36 of the screw shaft or along the shaft length, or both, such that the fulcrum member is past the pedicle and in the vertebral body when the screw assembly is engaged.

The longitudinal screw assembly 15 conforms to the size of a typical pedicle screw with the diameter of between approximately 5.5 mm and approximately 7.5 mm and length of between approximately 35 mm and approximately 50 mm. Furthermore, the screw assembly has design features that help maximize its pull out resistance, including, for example, mechanically operated anchor(s), balloon(s) or expansion member(s) that allow the screw to be fixed in place in the bone to provide good stability for the device 10 while the cutting wires are employed to cut surrounding bone.

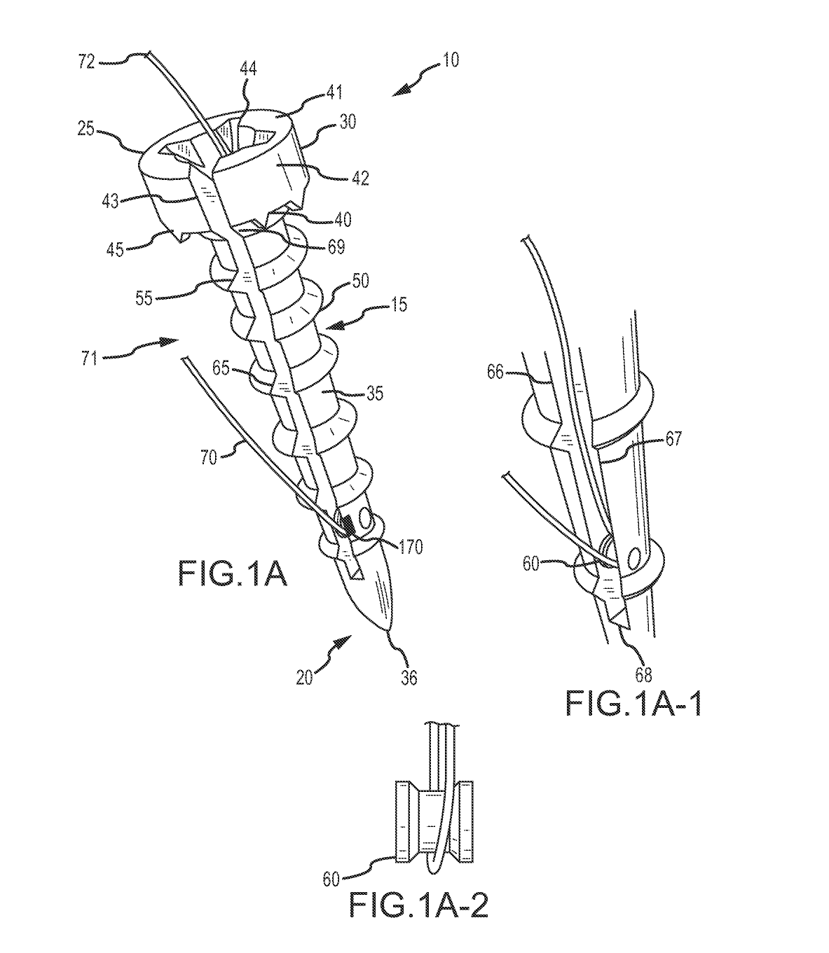

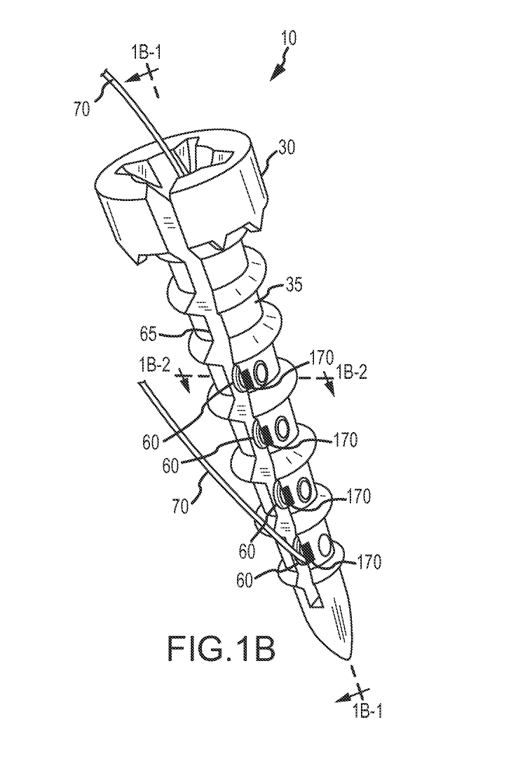

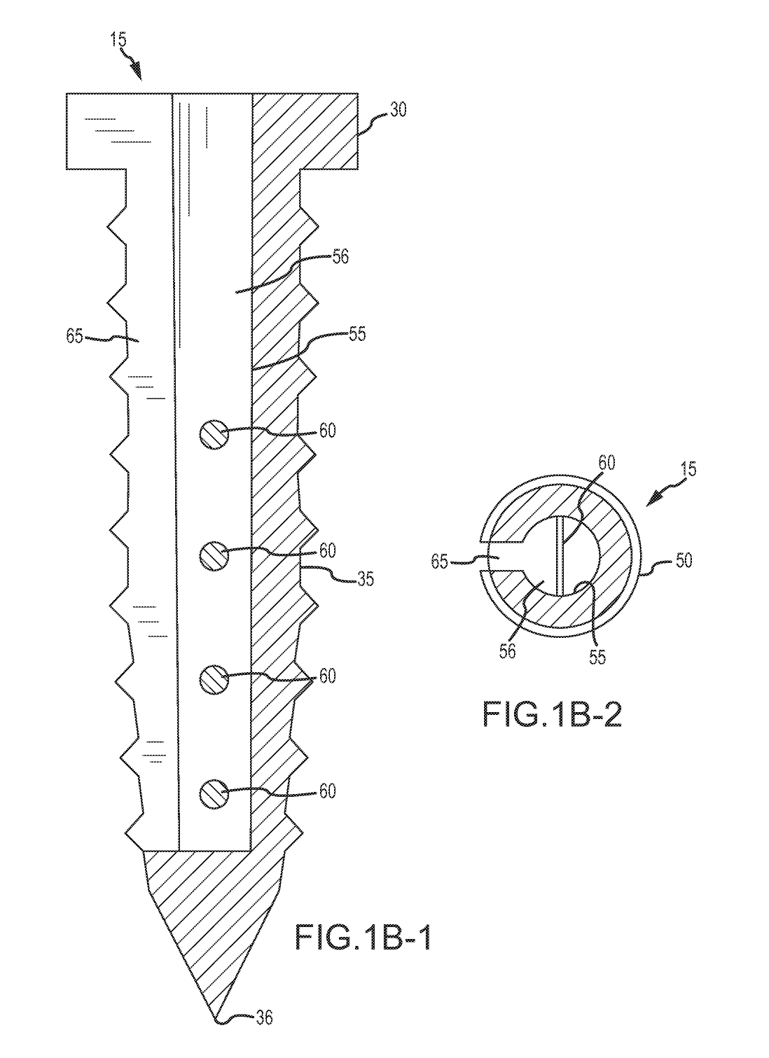

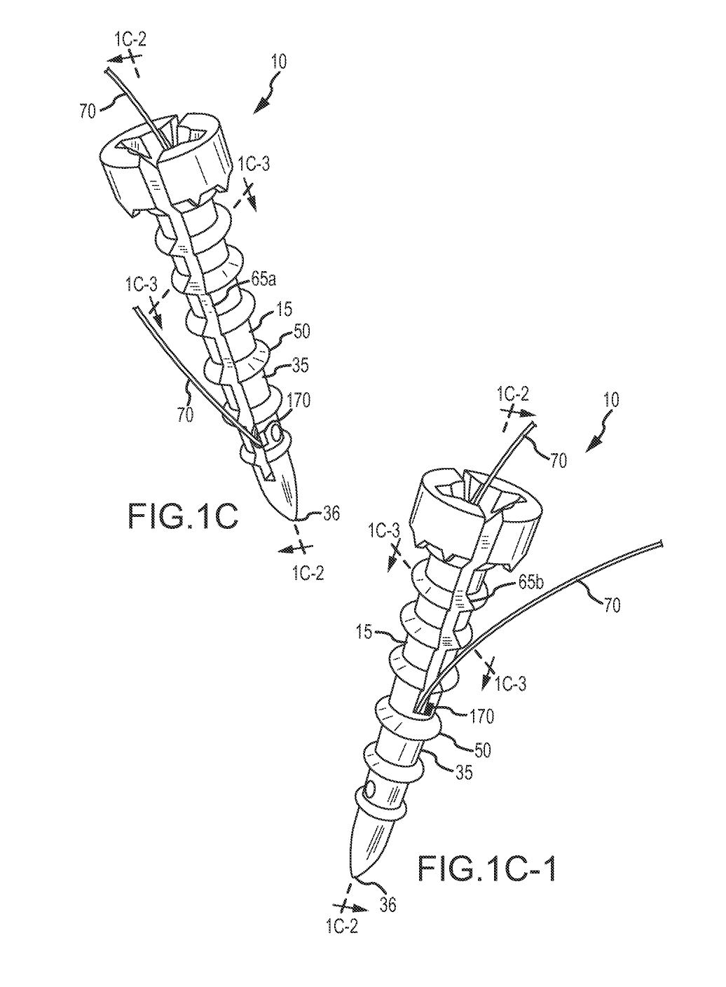

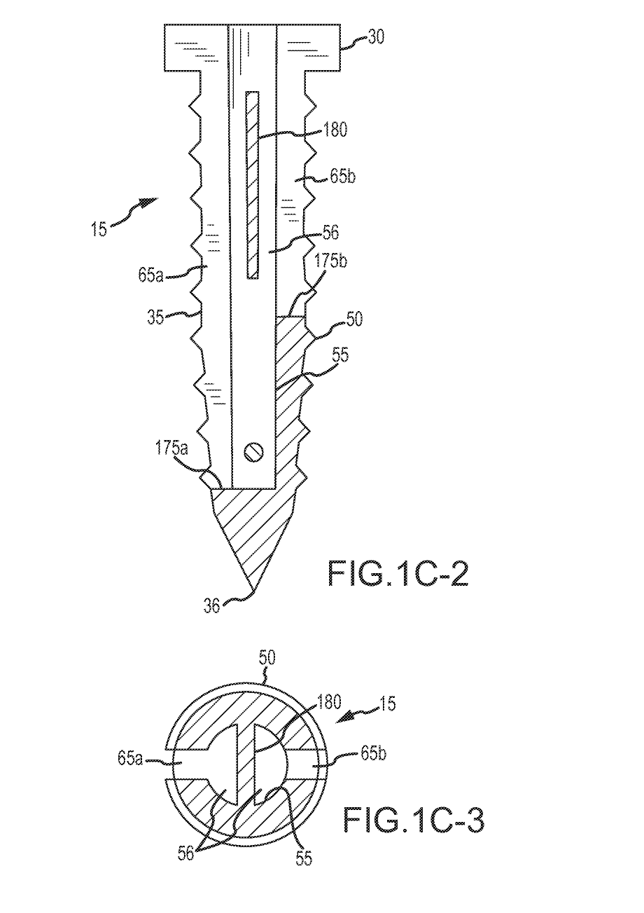

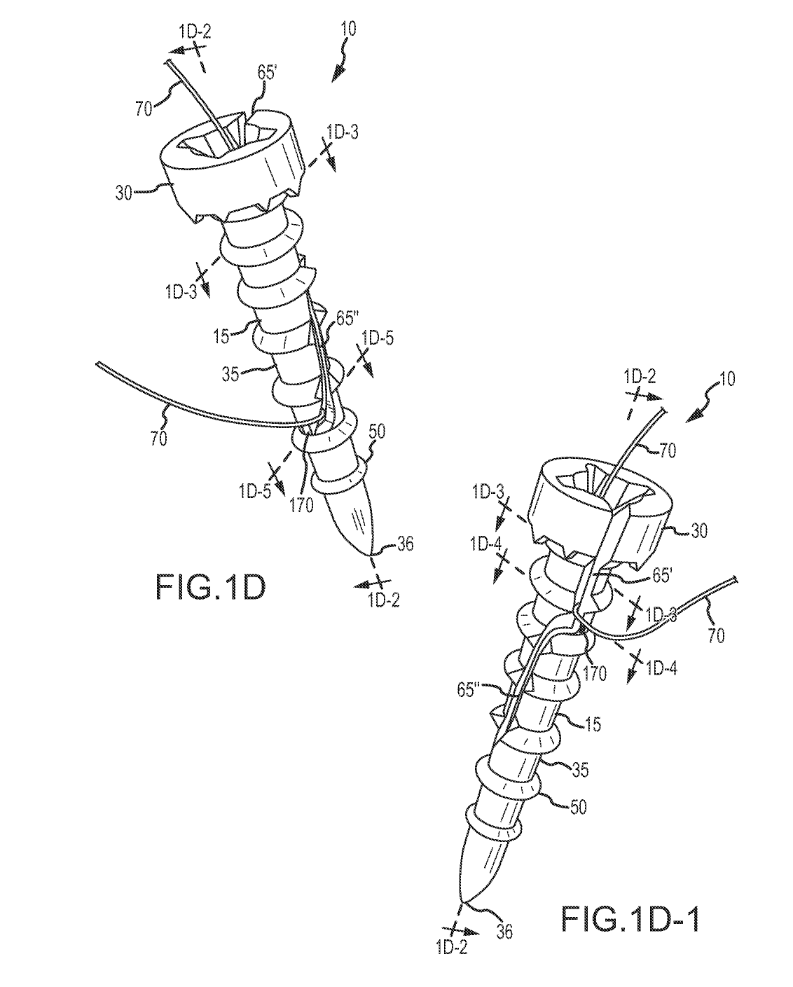

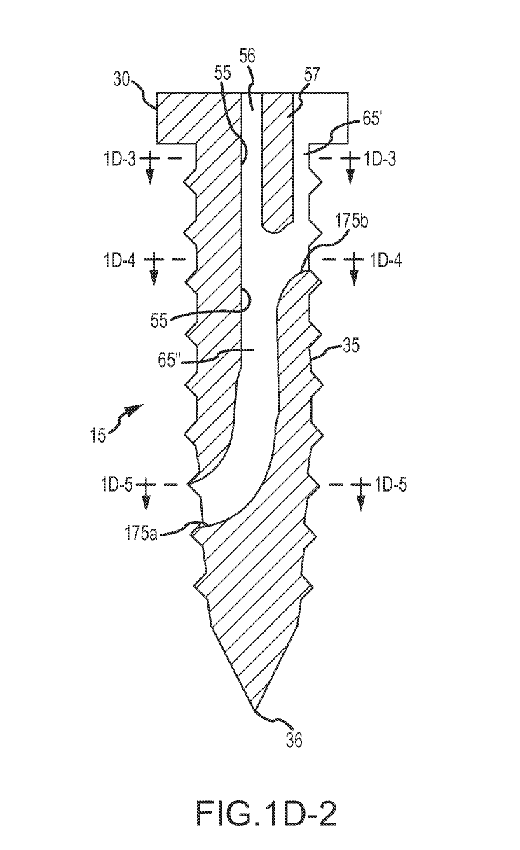

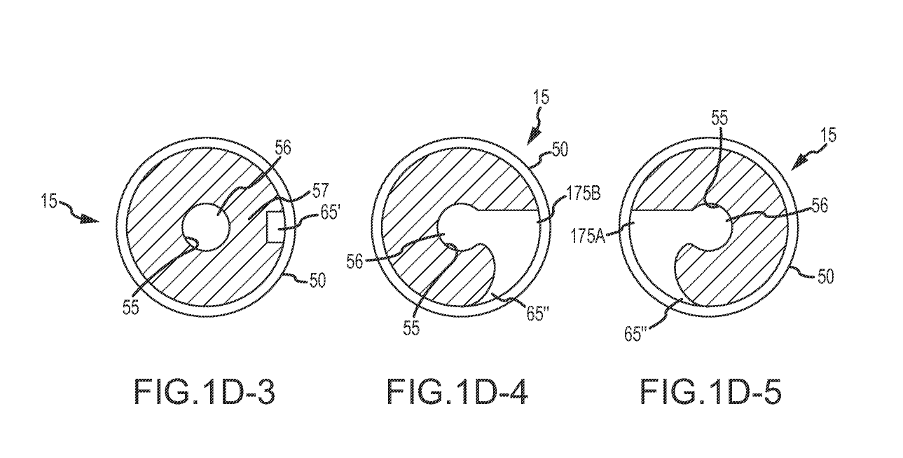

As indicated in FIGS. 1A, 1B and 1C, the screw assembly also includes one or more radio-opaque/radiographic markers 170 for identification of the exit point of the flexible cutting member 70 from the groove in the screw assembly via lateral fluoroscopy. The one or more grooves 65 through which the one or more flexible cutting member(s) 70 can exit start at the radio opaque marker that identifies the distal exit point of the flexible cutting member from the screw assembly. Thus, the radio opaque marker indicates the most distal location of the wire outside the screw and, under fluoroscopy, provides a positional indication to the physician that prevents the physician from pulling the flexible cutting member into the spinal canal.

The longitudinal screw assembly 15 is inserted into the vertebral body through a pedicle at the intended osteotomy site using surface landmarks, fluoroscopy and nerve monitoring. In one embodiment, once the screw assembly is engaged with the pedicle and the vertebral body, the flexible cutting member is routed into the screw and looped around the fulcrum. In another embodiment, the screw is engaged with the pedicle and vertebral body with the flexible cutting member already in the screw and looped around the fulcrum. Regardless of how the flexible cutting member ends up within the screw and looped about the fulcrum, with both ends of the flexible cutting member extending beyond the proximal end of the screw, handles are attached to the ends of the flexible cutting member using small latches or other attachment arrangements, such as for example, screws, clamps, or crimps. The cutting device 10 can be then used by manually directing one of the ends of the flexible cutting member out of the screw to engage a bone, while maintaining the other end of the flexible cutting member within the confines of the screw.

Because the cutting device 10 provides for more precise bone cuts, a more reliable correction of deformity and coronal imbalance can be accomplished. In other words, the use of the standard osteotomy techniques, which entail the use of drills, osteotomes and other tools, leads to imprecise bone cuts that can lead to unreliable correction of deformity, as well as multiple complications, such as bone bleeding or nerve injury. Thus, the device and methods disclosed herein are advantageous because they reduce complications that are associated with the currently employed tools and techniques, and increase predictability of the osteotomy procedure.

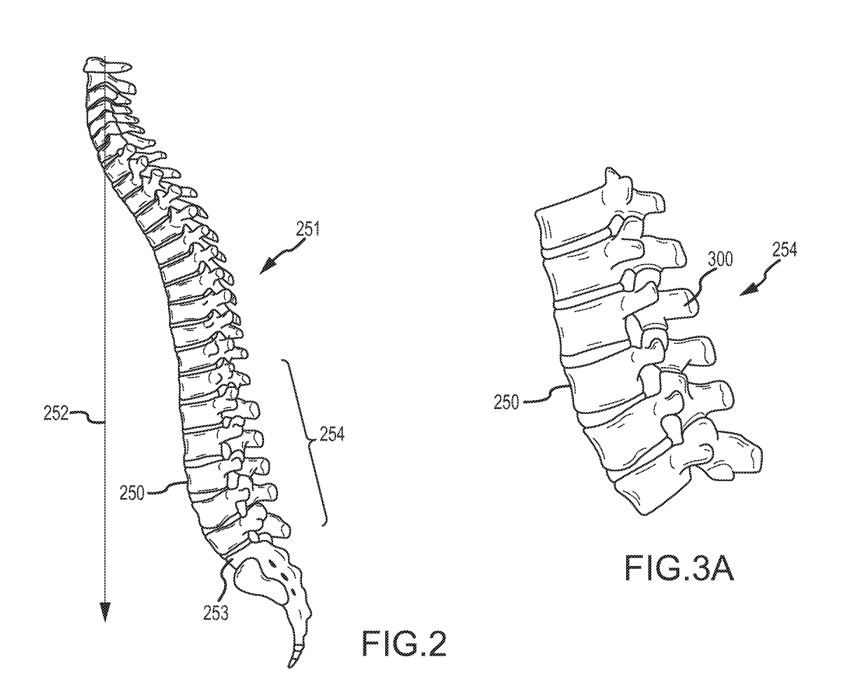

For a detailed discussion of the first embodiment of the device 10, reference is made to FIGS. 1A, 1A-1 and 1A-2, which are, respectively, an isometric view of a device intended for use in the pedicle subtraction osteotomy, an enlarged view of a distal region of the device of FIG. 1A, and a side view of a fulcrum supported in a distal region of the device of FIG. 1A. As shown in FIG. 1A, the device 10 includes a screw assembly 15 and a flexible cutting member 70 coupled with the screw assembly so as to allow the flexible cutting member to be used in a sawing motion as described in a greater detail below. The screw assembly has a distal end 20 and a proximal end 25 opposite the distal end. The screw assembly 15 also has a screw head 30 and a screw shaft 35.

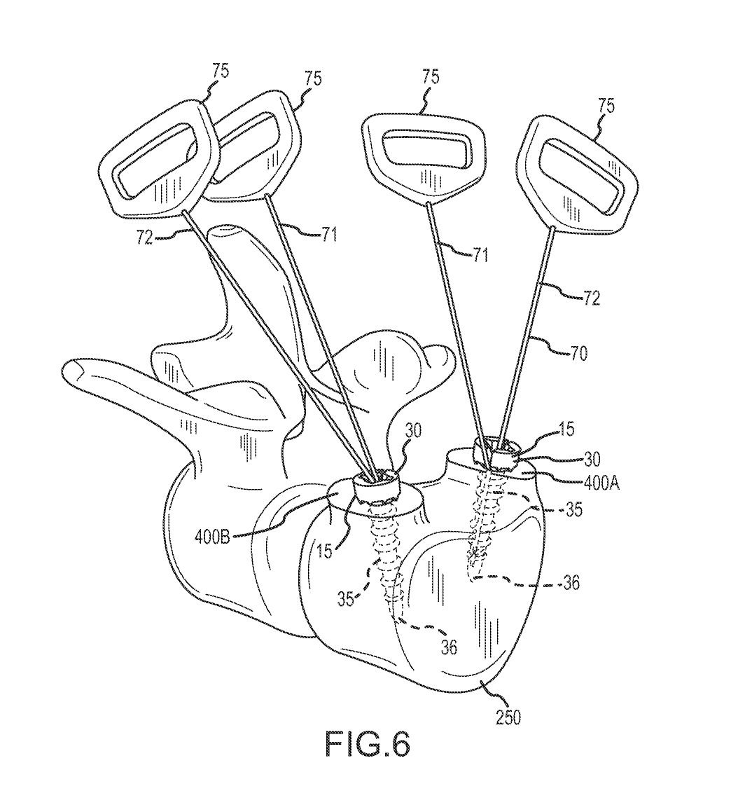

The screw shaft is an elongate, generally cylindrical component that extends distally from a distal face 40 of the screw head to a distal tip 36 of the screw shaft 35 at the distal end 20. The distal tip 36 may be tapered or smooth such that it does not interfere with the cutting wire 70. The screw shaft further includes screw threads 50 that cover substantially the entirety of the length of the screw shaft 35 between the distal face 40 of the screw head and the distal tip 36 of the screw shaft. The screw threads 50 of the screw shaft are constructed and adapted to engage and fix the screw assembly 15 within the vertebral body 250, as discussed below with respect to FIG. 6 below.

Furthermore, the central portion of the screw shaft is hollow, and defines an interior sidewall 55 of the screw shaft. In other words, a longitudinally directed cylindrical channel 56 (shown in FIGS. 1B-1 and 1B-2 discussed below) is formed inside the screw shaft 35 that is defined by an inner surface or an interior sidewall 55 through which flexible cutting members 70 can be navigated. Attached to the inner surface 55 of the screw shaft is a fulcrum member 60 that serves as a fulcrum and pivot or turning point for the one or more bone cutting flexible cutting member(s) 70. Specifically, the fulcrum member acts as a structural member about which the flexible cutting member is caused to turn a corner to result in the flexible cutting member having a V-shaped route having a first length extending between the fulcrum member and a first free end 71 of the flexible cutting member and a second length extending between the fulcrum member and a second free end 72 of the flexible cutting member. Further, the fulcrum member acts a pivot or displacement point that allows the flexible cutting member to displace about the fulcrum member as the flexible cutting member is displaced back and forth in a sawing motion by a medical professional grasping the flexible cutting member free ends 71, 72 via, for example, handles 75 coupled to the flexible cutting member free ends (e.g., see FIG. 6 discussed below). Finally, the fulcrum member 60 acts as a fulcrum that allows the flexible cutting member 70 to be levered or forced against the bone to be cut as the cutting wire 70 is displaced back and forth in the sawing motion.

In one embodiment, the fulcrum member 60 may simply be a shaft or other structural member extending across the longitudinally directed cylindrical channel 56 between opposed locations of the inner surface of the interior sidewall 55 and generally perpendicular to the longitudinal axis of the longitudinally directed cylindrical channel 56. Such a shaft 60 or other structural member may be rotatably or fixedly supported in the cylindrical channel.

In another embodiment, as illustrated in FIG. 1A-2, the fulcrum member 60 may be in the form of a pulley rotatably supported on an axel attached to the longitudinally directed cylindrical channel via the axel ends placed in opposed openings in the interior sidewall 55 of the longitudinally directed cylindrical channel

In other embodiments, the fulcrum member 60 can be a fixed pulley, a latch, or a groove, and can be located at or close to the distal tip 36 of the screw shaft, or along the screw shaft, or at any combination of these positions, as long as the fulcrum member is past the pedicle and in the vertebral body when the screw assembly is engaged. A radio opaque marker 170 may be located near the fulcrum to help the physician understand the location of the exit point of the flexible cutting member when the device 10 is imbedded in the vertebrae and viewed via fluoroscopy.

In one embodiment, as illustrated in FIG. 1A, the device 10 may have a single fulcrum member 60. In other embodiments as can be understood from FIGS. 1B through 1B-2, the device 10 may have multiple fulcrum members 60 extending between the interior sidewall 55 and spaced along the interior shaft 56. In such an embodiment, the flexible cutting member 70 can be caused to extend (e.g., threaded) about any one of the fulcrum members 60 so as to allow the “fulcrum” to be placed at any desired distance from the distal tip 36 of the screw shaft 35. The ability to place the “fulcrum” at different points along the length of the shaft provides the ability to cut to a pre-determined depth from the surface. A radio opaque marker 170 may be located near each fulcrum 60 to help the physician understand the location of the exit point of the flexible cutting member 70 when the device 10 is imbedded in the vertebrae and viewed via fluoroscopy.

As shown in FIGS. 1C through 1C-3, in other embodiments the screw 15 has multiple slots or grooves (e.g., two grooves 65a, 65b), each groove 65a, 65b having a distal terminus 175a, 175b with a unique distance from the distal tip 36. As a result of having grooves with different lengths, the point at which the flexible cutting member 70 exits the screw 15 can be adjusted between a deeper point on account of use of the longer slot 65a and its more distal terminus 175a or a more shallow point on account of use of the shorter slot 65b and its less distal terminus 175b. A radio opaque marker 170 may be located near the distal terminus of each slot 65a, 65b to help the physician understand the location of the exit point of the flexible cutting member 70 when the device 10 is imbedded in the vertebrae and viewed via fluoroscopy. As indicated in FIG. 1C-2, a structural reinforcement 180 may extend across the interior void or shaft 65 to provide additional structural stiffness to the screw 15 in addition to the structural stiffness provided by the screw structure near the tip 36.

In some embodiments, each radio opaque marker may be in the form of a fulcrum 60 made of a radio opaque material or a material that is substantially more radio opaque than the rest of the material forming the screw 15.

As shown in FIGS. 1D through 1D-5, in other embodiments the screw 15 has a single slot or groove 65 that includes a generally straight proximal segment 65′ and a spiral distal segment 65″. The straight segment 65′ extends distally through the screw head 30 and the most proximal portion of the screw shaft 35 to a first step or terminus 175b with a unique distance from the distal tip 36. The straight segment 65′ is separated from the interior shaft 56 by a shaft wall 57.

The spiral segment 65″ extends distally through the rest of the screw shaft 35 to a second step or terminus 175a with a unique distance from the distal tip 36. The spiral segment 65″ extends into or merges with the interior shaft 56.

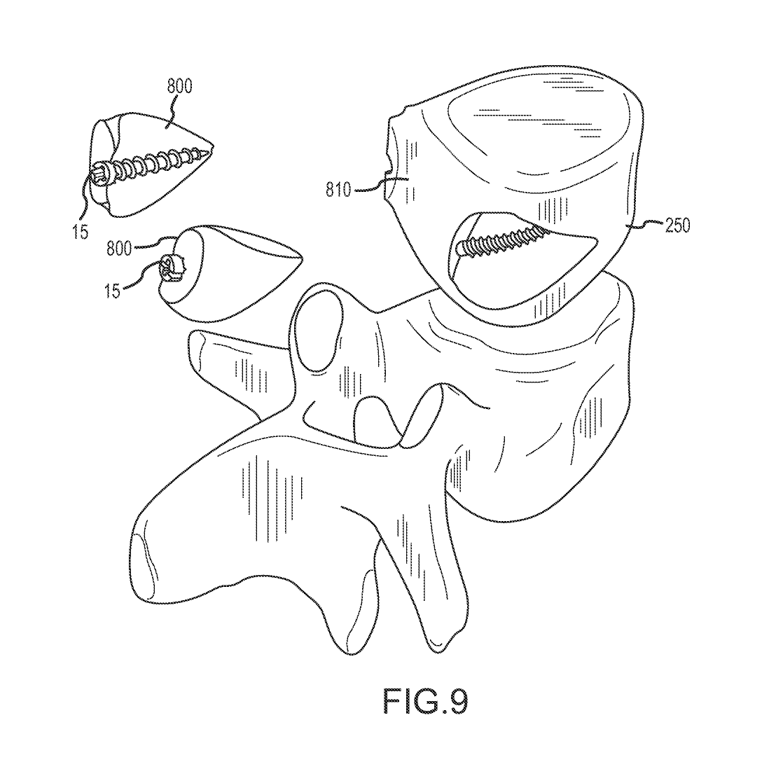

Each step or terminus 175a, 175b can act as a fulcrum having a different lever point from the distal tip 36. As a result of having a single groove 65 with different length steps 175a, 175b and a spiral section 65″ joining the two steps 175a, 175b, the point at which the flexible cutting member exits 70 the screw 15 can be adjusted between a deeper point on account of use of the most distal step 175a, a more shallow point on account of use of the most proximal step 175b, or any point between the most distal step 175a and the most proximal step 175b. Thus, the exit point of the flexible cutting member 70 can be caused to spiral around a side of the screw 15 between the two steps 175a, 175b. When the device 10 is imbedded in the bone such that the most proximal step 175b is oriented generally medial and the most distal step 175a is oriented generally lateral, the flexible cutting member 70 will exit the screw 15 more proximally such that the thecal sac will not be injured and, on the lateral side, the exit point would be more distal so more bone will be carved out, thereby facilitating a cut similar to that depicted in FIG. 9.

Radio opaque markers 170 may be located near the distal terminus 175a and the proximal terminus 175b to help the physician understand the location of the exit points of the flexible cutting member 70 when the device 10 is imbedded in the vertebrae and viewed via fluoroscopy.

In one embodiment as illustrated in FIGS. 1A and 1A-1, the screw shaft 35 further includes a longitudinally directed slot, channel, or groove 65 on the side of the screw shaft 35, which is defined by two opposed longitudinally extending side edges 66, 67 and proximal 69 and distal ends 68. In one embodiment, the groove 65 extends along substantially the entire shaft length of the shaft 35. However, as can be understood from the embodiments of FIGS. 1A through 1D-5, the groove can vary in length, shape and number (e.g., one or more longitudinal grooves can be present). As shown in these figures, the groove 65 extends directly into or is in communication with the hollow center of the screw shaft 56, such that the cutting wire 70 can be navigated through the central portion of the hollow shaft 35 to avoid bone cuts, or it can be allowed to exit the screw assembly through the groove and engage the bone.

As shown in FIG. 1A, the screw head 30 is constructed and adapted to be engaged by a tool such as, for example, a wrench or screw driver, to drive the screw shaft 35 into the bone when securing the device 10 in the bone or to withdraw the screw shaft 35 from the bone when removing the device from the bone. The screw head 30 includes a proximal face 41 and a cylindrical outer circumferential surface 42 extending between the proximal and distal faces 41, 40. The screw head further includes a longitudinally directed groove 43 on a side that extends into or is otherwise operationally connected to the groove 65 that extends along the side of the screw shaft 35. Also, an opening 44 in the middle of the screw head 30, which extends through the entire length of the screw head and which further connects to the hollow center 56 of the shaft 35 is present. In other words, the screw head is hollow, and the opening in the screw head is directly connected to the channel 56 in the screw shaft 35. Furthermore, in one embodiment a series of bone engaging features 45 can be equally spaced at the distal face 40 of the screw head 30 or around the circumferential surface 42 of the screw head. Alternatively, the distal face 40 of the screw head can be flat.

With reference still to the cutting device 10 shown in FIG. 1A, the discussion will now turn to the features of the flexible cutting member 70, which is employed to resect the bone. In particular, the cutting device 10 includes one or more flexible, elongated, thin-body cutting member(s) 70, which may take the form of a cutting wire, cable or rope and will be generically referred to herein as flexible cutting member 70. The flexible cutting member is of a predetermined length and has a first free end 71 and a second 72 free end. Furthermore, cutting features (e.g., teeth) or abrasive features (e.g., imbedded grit or hard rough particles) are present along substantially the entire length of the flexible cutting member that extends between the first and the second free ends. In some embodiments, the cutting features or abrasive features of the flexible cutting member 70 may be only along a limited segment of the flexible cutting member (e.g., along the middle third or half of the flexible cutting member) while the remainder of the flexible cutting member 70 is relatively smooth and free of cutting or abrasive features.

The flexible cutting member 70 is introduced into the screw assembly 15 by threading one of its free ends 71 or 72 through the opening 44 in the middle of the screw head 30 at the proximal end 25 of the screw assembly. The flexible cutting member 70 then runs along the hollow center 56 of the shaft 35 until it reaches the fulcrum member 60. At the fulcrum member the flexible cutting member loops about or through the fulcrum member and runs back to the proximal end 25 of the screw assembly 15, along the hollow center 56 of the shaft 35. More specifically, the flexible cutting member 70 is routed through the hollow shaft 35, and about or through the fulcrum member 60, such that it assumes a folded or V-shaped configuration, having a first length extending between the fulcrum member and a first free end 71 of the flexible cutting member and a second length extending between the fulcrum member and a second free end 72 of the flexible cutting member 70.

Once the flexible cutting member 70 is routed through the screw assembly 15 and both of its free ends extend beyond the proximal end of the screw 15, a handle 75 is attached to each free end 71, 72 of the cutting wire 70 using small latches. Depending on the embodiment, the handles 75 may be in the form of T-handles, rings, spherical members, or any other configuration that allows a medical professional to securely grasp the handles.

In some embodiments, the flexible cutting member 70 may be in the form of a gigli saw. In some embodiments, the flexible cutting member is in the form of a wire, cable, thread or rope material formed of a metal or synthetic material and having cutting aspects in the form of teeth or an imbedded or coated abrasive.

For a discussion of a method of employing the cutting device 10 in the pedicle subtraction osteotomy procedure, reference is now made to FIGS. 2 through 15. In FIG. 2 a sagittal view of a deformed spine 251 of a patient scheduled to undergo a pedicle subtraction osteotomy is shown. In particular, the center of the gravity 252 of a deformed spine (which is depicted in the figure by an arrow) is shown as being located in front of the sacrum 253. The goal of the surgery, thus, is to restore sagittal balance by returning the center of the gravity to fall through the sacral promontory as shown in FIG. 15.

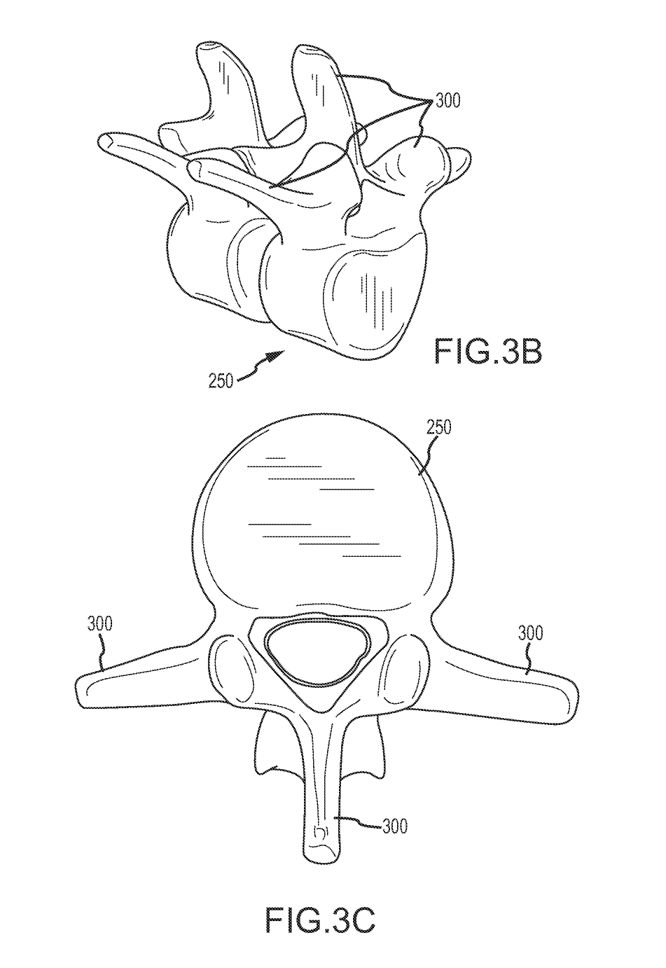

To begin the procedure, a segment of the spine 254 on which osteotomy is to be performed is first selected, as indicated in FIG. 2. FIGS. 3A-3C are, respectively, sagittal, superior-sagittal isometric, and superior views of the selected spine segment 254, prior to the commencement of the procedure, with the vertebra 250 still intact. FIGS. 3A-3C, show the vertebra 250 prior to the removal of both the lamina 300 and facets.

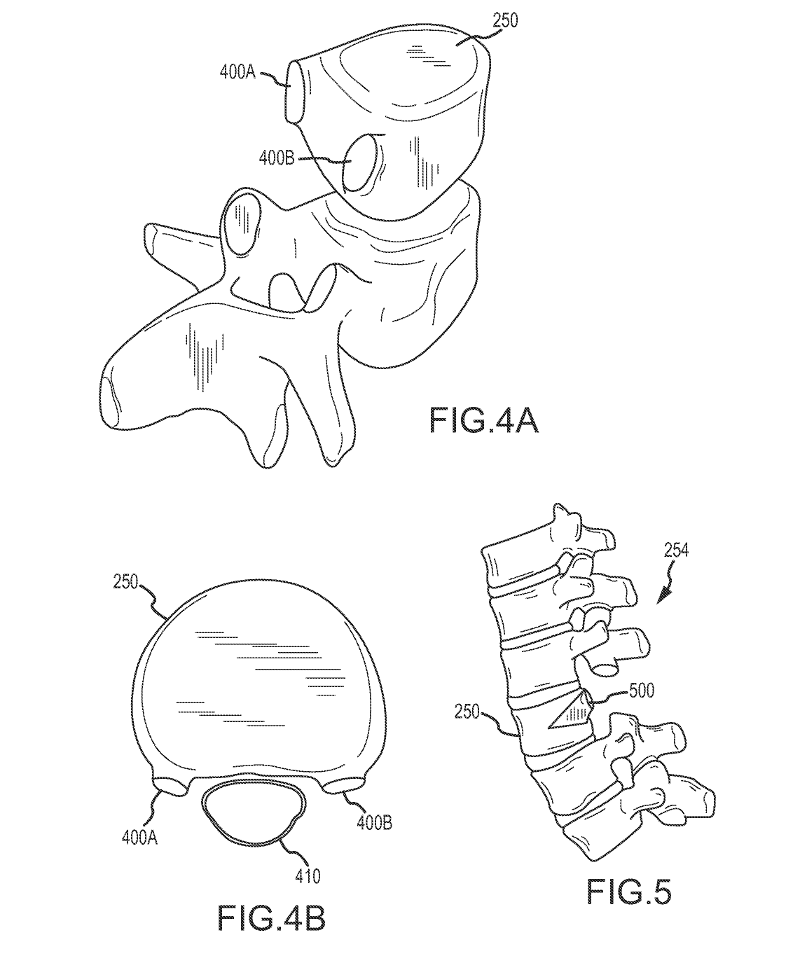

FIGS. 4A and 4B are, respectively, superior-posterior isometric and superior views of the vertebra 250 following the laminectomy and facetectomy. As can be understood from FIGS. 4A and 4B, the laminectomy and facetectomy expose pedicles 400, and both nerve roots (not shown) and thecal sac 410. Once the pedicles, and both nerve roots and thecal sac are exposed, the osteotomy site 500 may be further identified and inspected, as can be understood from FIG. 5, which is the same view as FIG. 3A, except subsequent to the laminectomy and facetectomy.

In FIG. 6, which is the same view as FIG. 3B, except subsequent to the laminectomy and facetectomy, each screw assembly 15 is inserted into the vertebral body through a respective pedicle 400A, 400B. More specifically, each threaded shaft 35 of the screw assembly 15 is threadably driven into the vertebral body using a tool such as, for example, a wrench or screw driver that can drive the screw shaft into the bone of the respective pedicle. Surface landmarks, fluoroscopy and nerve monitoring are used during the insertion process in order to ensure the correct placement of the screw assembly. The screw assembly 15 is driven into the pedicle 400 until the distal face 40 of the screw head is flush against the bone surface. In another embodiment, the screw assembly 15 is driven into the pedicle 400 until the bone engaging features 45, enter the bone. Once the screw assembly is anchored in the pedicle and vertebral body 250, a flexible cutting member 70 is threaded through the screw assembly and handles 75 are attached. For example, in one embodiment, with the screw 15 already fully anchored in the vertebral body 250, a free end 71 of the flexible cutting member 70 is threaded down through the cylindrical channel 56 and around the fulcrum 60 and back up through the channel 56 such that the cutting member 70 bends about the fulcrum 60 as both free ends 71, 72 extend from the cylindrical channel 56 where the cylindrical channel daylights in the screw head 30. As indicated in FIG. 6, the handles 75 are then secured to the respective free ends 71, 72 of the flexible cutting member 70.

In another embodiment, prior to the screw 15 the being anchored in the vertebral body 250, a free end 71 of the flexible cutting member 70 is threaded down through the cylindrical channel 56 and around the fulcrum 60 and back up through the channel 56 such that the cutting member 70 bends about the fulcrum 60 as both free ends 71, 72 extend from the cylindrical channel 56 where the cylindrical channel daylights in the screw head 30. With the flexible cutting member 70 so preloaded in the screw 15, the screw 15 is then driven into the vertebral body 250 until the screw is fully anchored in the vertebral body. To facilitate such an anchoring of the preloaded screw into the vertebral body, a driving tool (e.g., wrench or screw driver) with a hollow shaft may be employed to drive the screw 15 into the vertebral body. In doing so, the free ends 71, 72 of the preloaded flexible cutting member 70 are routed up through the hollow shaft of the driving tool and the distal end of the hollow shaft of the driving tool is then engaged with the screw head 30 to drive the screw 15 into the vertebral body with the flexible cutting member 70 preloaded in the screw 15 about the fulcrum 60 and extending through the cylindrical channel 56 of the screw. Once the screw is fully anchored in the vertebral body 250 as illustrated in FIG. 6, the driving tool is removed and the handles 75 are secured to the respective free ends 71, 72 of the flexible cutting member 70.

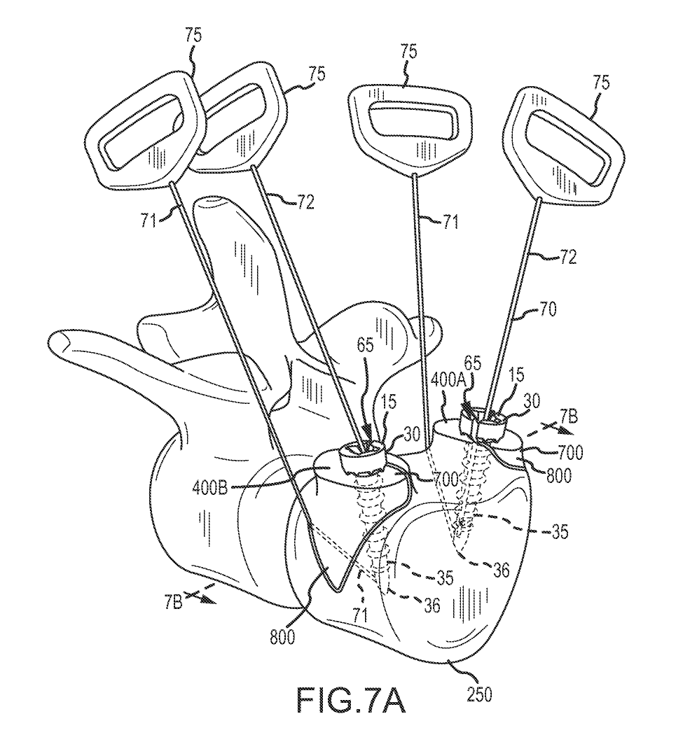

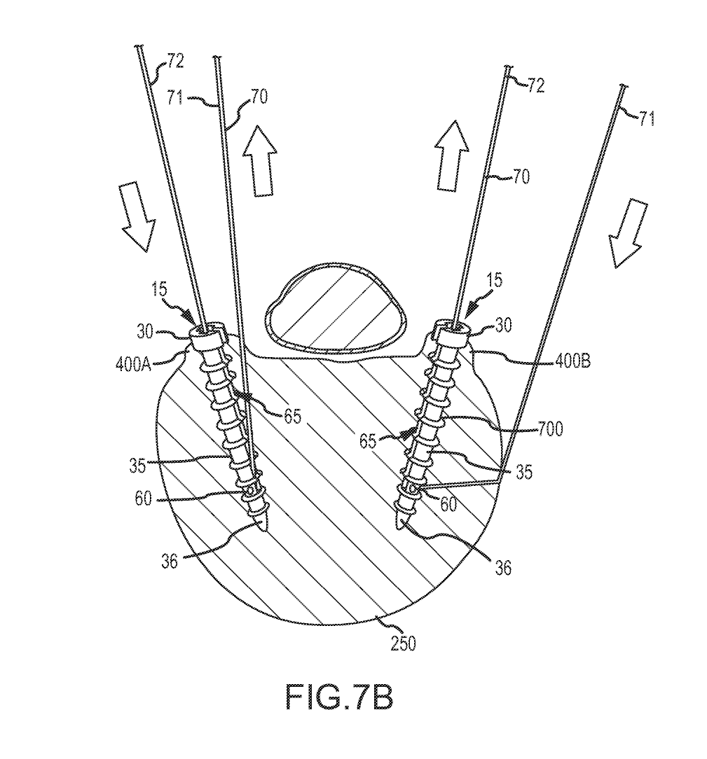

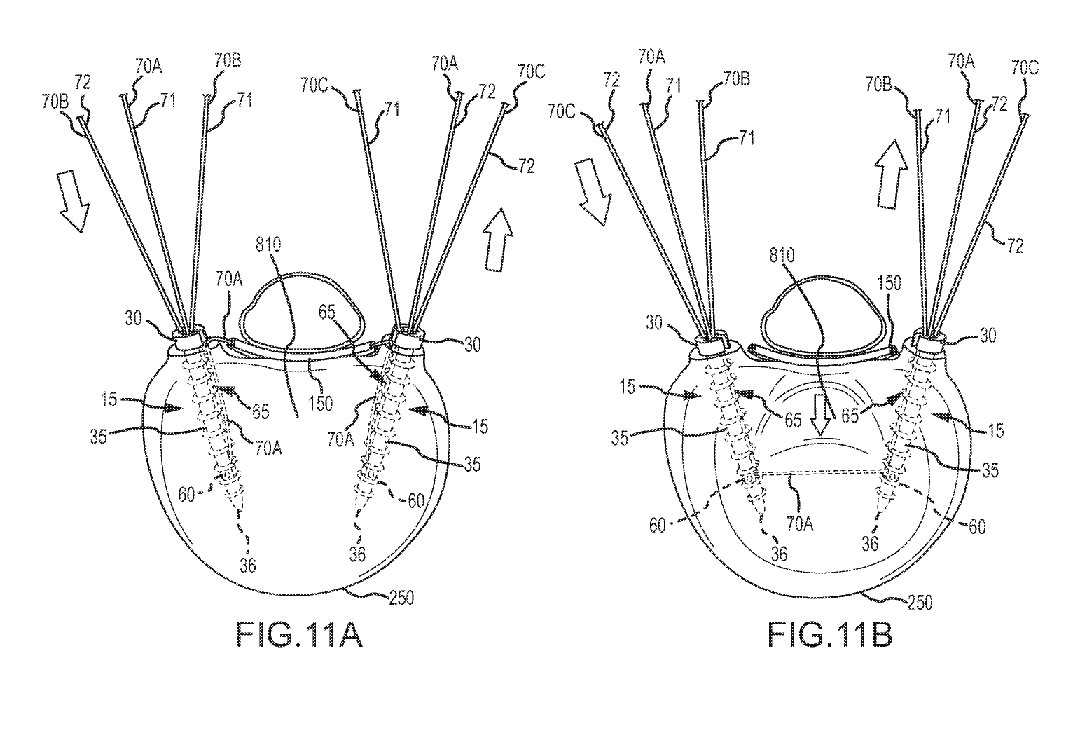

FIGS. 7A and 7B will now be referred to in beginning a discussion of resecting the bone via the device 10, wherein FIG. 7A is the same view as FIG. 6 and FIG. 7B is a cross section taken along section line 7B-7B in FIG. 7A. As depicted in the FIGS. 7A and 7B, the bone sawing process begins with manually directing one of the lengths 71 of the flexible cutting member 70 out of the screw assembly 15 through a groove 65 on the side of the screw shaft 35 in order to engage the bone, while maintaining the other length 72 of the flexible cutting member 70 within the confines of the screw 15. In other words, one of the lengths 71 of the flexible cutting member 70 extending between the fulcrum member 60 and the one of the free ends 71 of the flexible cutting member 70 is guided to the outside of the screw assembly 15 in order to engage the bone, while the second length 72 of the flexible cutting member 70 extending between the fulcrum member 60 and the second free end 72 remains inside the screw assembly 15. By allowing one length 71 of the flexible cutting member 70 to exit the screw assembly 15, while maintaining the other length 72 of the flexible cutting member 70 within the confines of the screw assembly 15, a controlled bone cut can be achieved. As can be understood from the figures, a metal or plastic barrier 150 can be used to protect the ventral dura. Such a barrier 150 can be placed at the beginning of the procedure if passing a wire 70 under the ventral dura to make the center cuts as indicated in FIGS. 11A and 11B, for example.

As shown in FIGS. 7A and 7B, the cephalad portion 700 of the pedicle 400A, 400B is sawn through first. The pedicle sawing is accomplished by moving the flexible cutting member 70 back and forth in a sawing motion as depicted by the arrows in FIG. 7B. More specifically, by levering or forcing the flexible cutting member 70 against the bone to be cut, and at the same time displacing the flexible cutting wire 70 about the fulcrum member 60 in the back and forth sawing motion, a controlled cut through the pedicle 400A, 400B is made. Once, the cephalad portion 700 of the pedicle is sawn through, the sawing process continues by guiding the flexible cutting member 70 in a circumferential fashion through both the pedicle 400A, 400B and vertebral body 250, using the sawing motion described above. In particular, the flexible cutting member 70 is navigated through both the pedicle and vertebral body using the back and forth sawing motion in a circumferential fashion in order to form pedicle and vertebral body bone wedges or blocks 800. In one embodiment, a mechanically operated expansion mechanism that can exert pressure on the sides of the pedicles can be used to provide additional stability if the bone surrounding the screw 15 loosens as the wire 70 compromises surrounding bone.

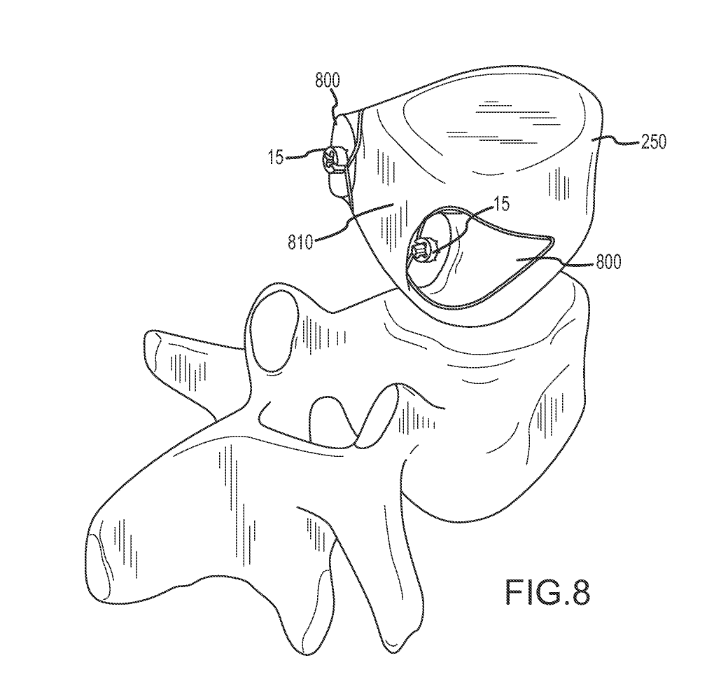

As shown in FIG. 8, for each screw 15 anchored in each respective pedicle 400A, 400B, a simple circumferential pass of a single flexible cutting member 70 results in a formation of a bone wedge or block 800 at each respective pedicle, with a central piece 810 of the vertebra bone, under the dura still intact. The screw assembly 15 and bone wedges or blocks 800, according to one embodiment shown in FIG. 9, can be removed en block, followed by a piecemeal removal of the central piece 810 of the vertebra bone with the standard instruments.

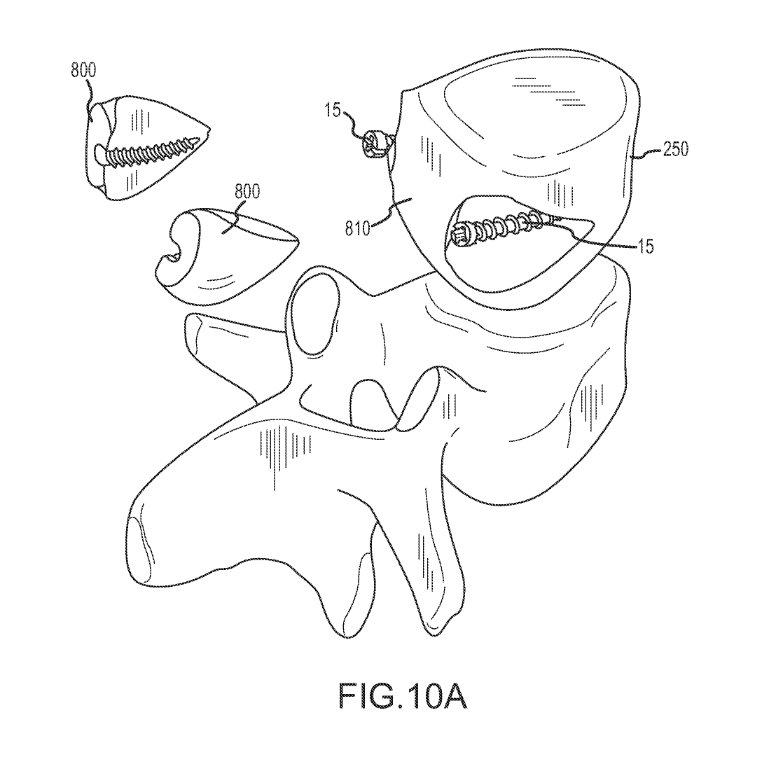

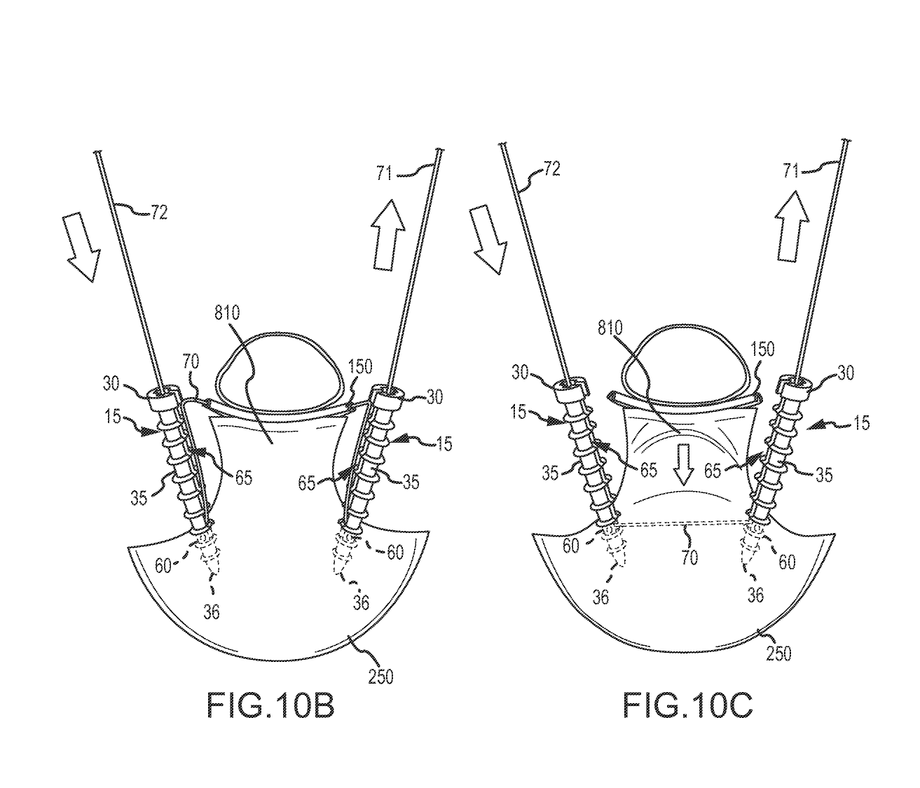

In accordance with another embodiment, as shown in FIG. 10A, following the bone resection only the sawn out bone wedges or blocks 800 are removed leaving the screw assembly 15 in both the right and left pedicles of the vertebral body 250 still engaged. In order to remove the central piece 810 of the vertebra 250, a second flexible cutting member 70 is used. More specifically, once the bone wedges 800 are sawn out as discussed above with respect to FIGS. 6-8 and then removed, a second flexible cutting member 70 is inserted thought a screw assembly 15 in either the right or left pedicle of the vertebral body 250. As shown in FIG. 10B, the second flexible cutting member 70 is then guided under the ventral dura to the second screw assembly 15 still engaged in the other pedicle of the vertebral body. Once the second flexible cutting member 70 is threaded through both the right and left screw assemblies 15, the central vertebral bone 810 can be then sawn by moving the second flexible cutting member 70 in an up and down sawing motion as shown by the arrows in FIGS. 10B and 10C. As indicated in FIGS. 10B and 10C, a shield or guard 150 may be located between the ventral dura and the flexible cutting member 70 to protect against the flexible cutting member from damaging nerve tissue.

In yet another embodiment, shown in FIGS. 11A and 11B, two flexible cutting members 70A, 70B, and 70C per screw can be used. In other words, in one embodiment, each screw 15 has its own respective flexible cutting member and a second flexible cutting member that is shared between the two screws, resulting in a total of three flexible cutting members and two screws. In another embodiment, each screw 15 has a first flexible cutting member and a second flexible cutting member. The second flexible cutting member of the left screw is joined with the second flexible cutting member of the right screw to form a single flexible member that is shared between the two screws, again resulting in a total of three flexible cutting members and two screws.

As can be understood from FIGS. 11A and 11B, whether starting out as a single shared flexible cutting member or as a pair of flexible cutting members that are joined to form a single shared flexible cutting member, such a shared flexible cutting member 70A extends between the two screws 15 with one free end 71 extending about the fulcrum 60 of the left screw 15 and out the left screw to a first handle, and the other free end 72 extending about the fulcrum 60 of the right screw 15 and out the right screw to a second handle. Another flexible cutting member 70B extends about the fulcrum 60 of the left screw 15 such that both ends 71, 72 of the flexible cutting member 70B extend out of the left screw to respective handles. Similarly, yet another flexible cutting member 70C extends about the fulcrum 60 of the right screw 15 such that both ends 71, 72 of the flexible cutting member 70B extend out of the right screw to respective handles.

The flexible cutting member arrangement depicted in FIGS. 11A and 11B is to allow for the removal of pedicle and vertebra body, as well as the central vertebral bone. Thus, according to this embodiment, a first flexible cutting member 70A of the right screw 15 is used to cut through the cephalad pedicle on right side of the vertebra. Similarly, a first flexible cutting member 70A of the left screw 15 is used to cut through the cephalad pedicle on left side of the vertebra. Following the initial cut, the first flexible cutting member 70A of the right screw 15 is passed through the under the ventral dura. The right and left first flexible cutting members 70A, 70A are connected with a small latch mechanism to form a shared flexible cutting member 70A that extends between the two screws 15 with one free end 71 extending about the fulcrum 60 of the left screw 15 and out the left screw to a first handle, and the other free end 72 extending about the fulcrum 60 of the right screw 15 and out the right screw to a second handle. With ventral dura being protected with a metal or plastic barrier, shield or guard 150, the first flexible cutting member 70A is used to saw through the posterior canal cortex and vertebra down to the screw tips, as can be seen via the progression illustrated in FIGS. 11A-11B.

A latch mechanism on a screw tip of one of the screws 15 is used to allow the first flexible cutting member 70A to flip from cephalad to caudal, and then the bone is sawed from the screw tip to near (but not through) the posterior cortical line on lateral fluoroscopy. Alternatively, the groove through which the wire 70A exits the screw 15 can have a stop to prevent the wire from pulling up into the spinal canal. In some embodiments, the right and left wires 70A forming the first shared wire 70A can be connected via, for example, a latch similar to a bracelet latch or other latching arrangements.

Once the central vertebral bone 810 is cut through, the second wires 70C, 70B is used to circumferentially remove the right and left pedicles respectively, as previously described. The bone blocks or wedges 800 are then manually removed to complete the osteotomy. In one embodiment, both right and left screws 15 are used as fulcrums for both wires. In such configuration, a pulley 60 can be used to reduce resistance that results from having both flexible cutting members threaded through the fulcrums 60 concurrently.

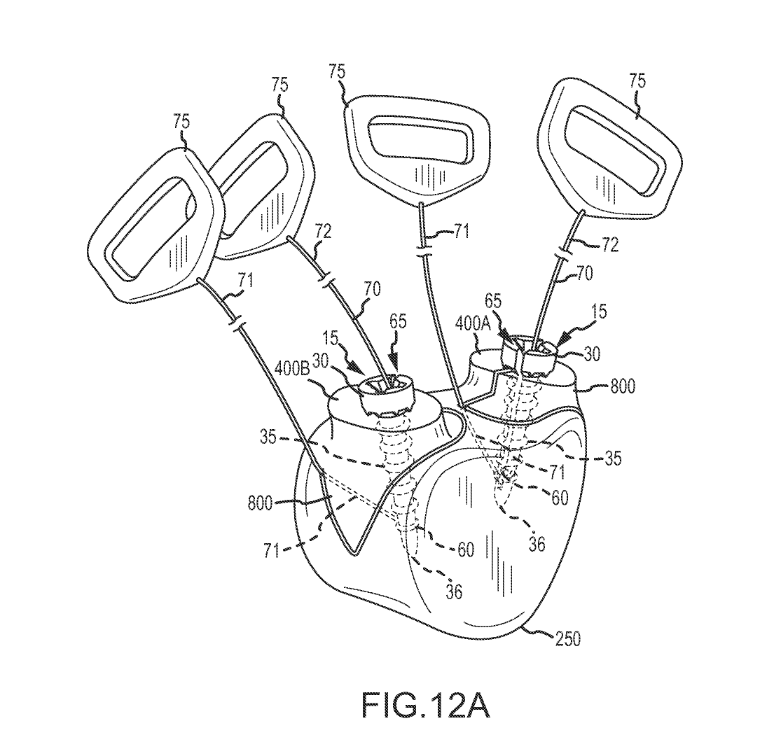

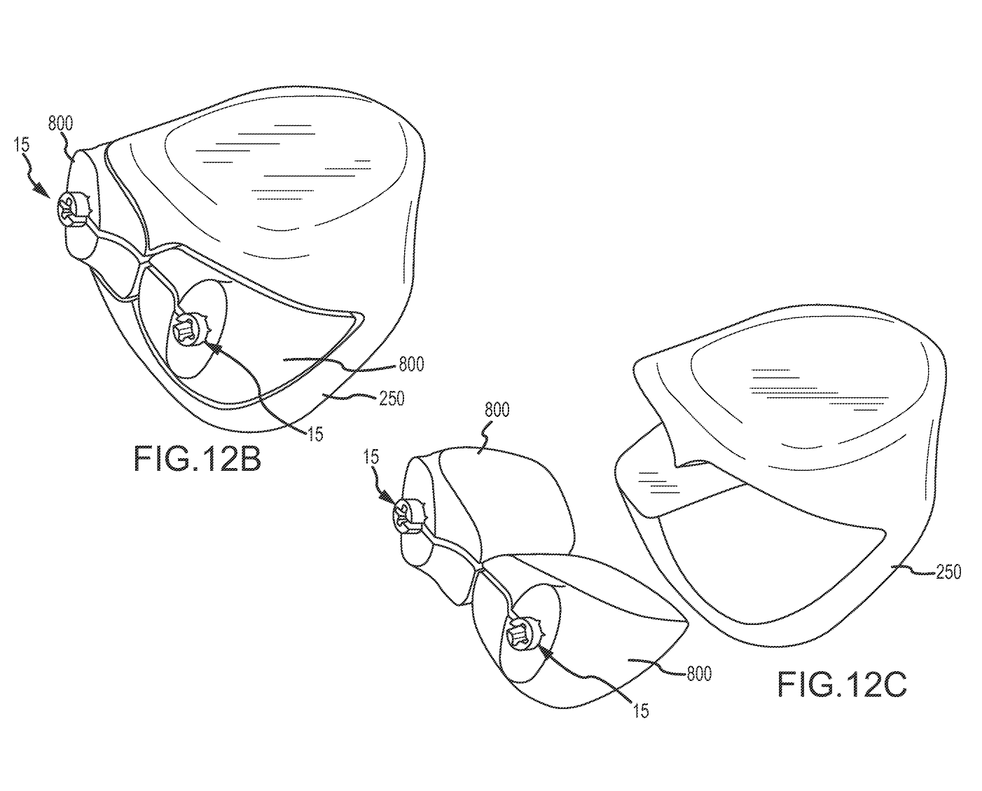

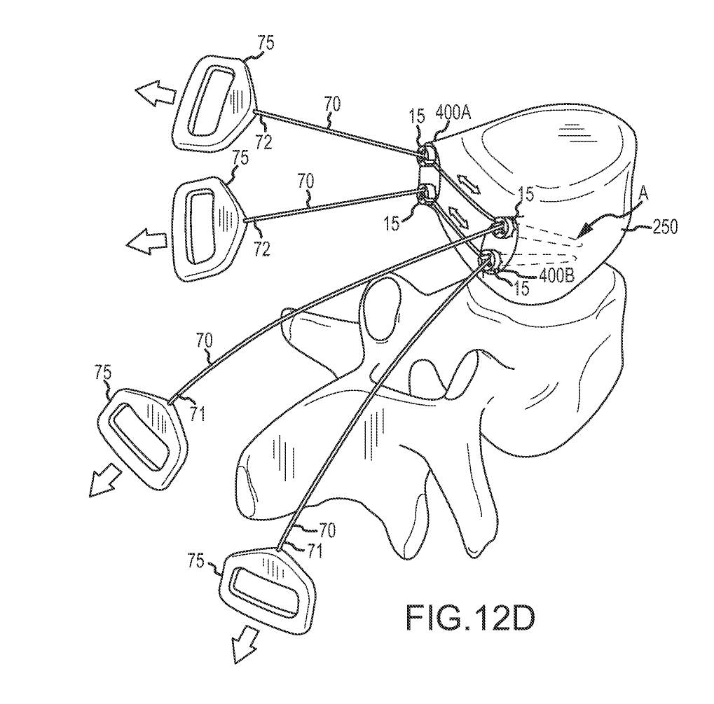

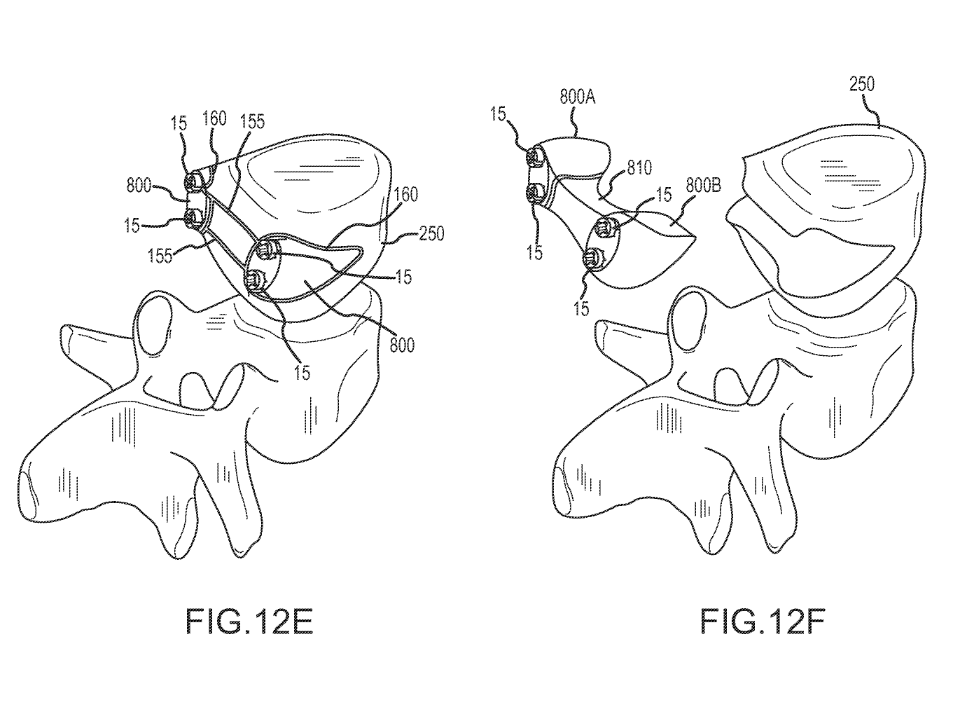

In another embodiment shown in FIGS. 12A-12B, a single flexible cutting 70 member is used in each of the screw assemblies 15 engaged in the right and left pedicles 400B, 400A. In such an embodiment a single circumferential pass of each of the flexible cutting members 70 causes a resection of both the pedicle and vertebral body, as well as the central vertebral bone as shown in FIG. 12B. The resected wedges or blocks 800 formed during the single pass of the flexible cutting member on both the right and left side can be then removed en block along with the screw assemblies 15 as is depicted in FIG. 12C.

In yet another embodiment shown in FIGS. 12D-12F, two screw assemblies 15 are engaged in each of the right and left pedicles 400B, 400A. As indicated by arrow A in FIG. 12D, each set of screw assemblies 15 engaged in a pedicle have the screw assemblies 15 oriented in the pedicle such that the distal tips of the screw assemblies converge and the proximal ends of the screw assemblies diverge. As a result, the two screw assemblies in the respective pedicles form generally a V-arrangement relative to each other when viewed sagitally.

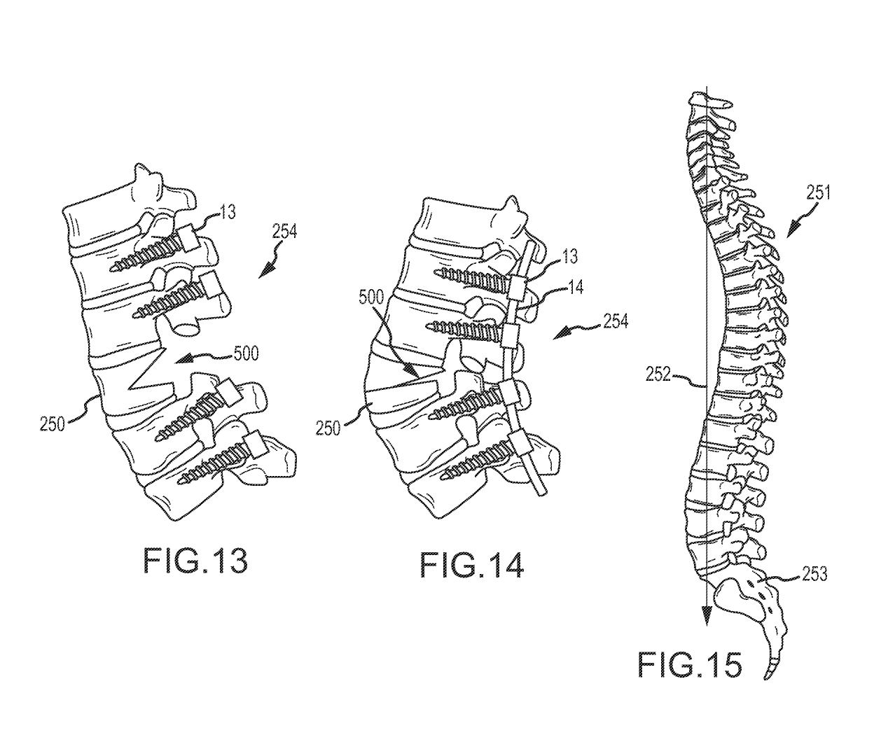

As shown in FIG. 12D, a single flexible cutting member 70 extends medial-lateral between each screw assembly 15 of the upper pair of screw assemblies, and another single flexible cutting member 70 extends medial-lateral between each screw assembly of the lower pair of screw assemblies. With the flexible cutting members 70 arranged as shown in FIG. 12D and a guard 150 provided between each flexible cutting member 70 and the ventral dura as depicted in FIG. 10B, upper and lower medial horizontal cuts 155 can be made in the vertebrae as illustrated in FIG. 12E. A handle 75 can then be decoupled from one end of each flexible cutting member and recoupled to its respective end after each flexible cutting member is set up to operate out of a single screw assembly in a manner similar to that shown in FIG. 12A, thereby allowing each flexible cutting member to make a circumferential cut 160 as indicated in FIG. 12E. The result of the horizontal cuts 155 and circumferential cuts 160 may be three bone portions such as a right pedicle portion 800B, left pedicle portion 800A and an intermediate or center portion 810, as illustrated in FIG. 13F. The three resected wedges or blocks 800A, 800B, 810 formed during the passes of the flexible cutting member discussed with respect to FIGS. 12D and 12E can be then removed en block along with the screw assemblies 15 as is depicted in FIG. 12F.

Once the osteotomy is completed following the above described method, reduction and instrumentation using standard techniques is performed to finish the procedure. In particular, as shown in FIG. 13, which is a sagittal view of the spine segment 254 being treated, four pedicle screws 13 are inserted in the pedicles surrounding the osteotomy site 500. As illustrated in FIG. 14, a wire 14 is threaded through the heads of the pedicle screws 13 in order to connect all of the screws. More specifically, by tying together the four screws, the gap created by the osteotomy can be closed, and as such allowing for shortening of the posterior column. In the end, a successfully performed procedure will lead to the restoration of the sagittal balance so that the center of gravity 252 will once again fall through the sacral promontory of the sacrum 253, as shown in FIG. 15.

The foregoing merely illustrates the principles of the various embodiments of the systems, devices and methods disclosed herein. Various modifications and alterations to the described embodiments will be apparent to those skilled in the art in view of the teachings herein. It will thus be appreciated that those skilled in the art will be able to devise numerous systems, devices and methods which, although not explicitly shown or described herein, embody the principles of the systems, devices and methods disclosed herein and are thus within the spirit and scope of the present disclosure. From the above description and drawings, it will be understood by those of ordinary skill in the art that the particular embodiments shown and described are for purposes of illustration only and are not intended to limit the scope of the claims associated with the present disclosure. References to details of particular embodiments are not intended to limit the scope of the claims associated with the present disclosure.

What is claimed is:

1. A device for cutting bone in performing a pedicle subtraction osteotomy, the device comprising:
an elongated member comprising a distal end, a proximal end, an elongated slot between the distal end and proximal end and extending proximal-distal, and a fulcrum located distal the proximal end; the elongated member configured to be anchored to the bone, and
a flexible cutting member extending through the elongated slot and around the fulcrum such that at least a portion of the flexible cutting member can extend from the elongated slot to cut the bone as the flexible cutting member is moved back and forth in a sawing fashion about the fulcrum.

2. The device of claim 1, wherein the elongated member comprises a threaded bone screw.

3. The device of claim 1, wherein the flexible cutting member comprises an abrasive or tooth equipped wire, cable or rope.

4. The device of claim 1, wherein the elongated slot in extending proximal-distal extends generally parallel to a longitudinal axis of the elongated member.

5. The device of claim 1, wherein the proximal end comprises a head configured for screwdriver or wrench engagement.

6. The device of claim 5, wherein the elongated slot extends through a side of the head.

7. The device of claim 1, wherein the elongated member further comprises a channel extending along a longitudinal axis of the elongated member, the elongated slot opening into the channel.

8. The device of claim 7, wherein, in addition to the flexible cutting member extending through the elongated slot and around the fulcrum, the flexible cutting member also extends through the channel to exit out the proximal end.

9. The device of claim 1, wherein the elongated member comprises a threaded outer surface.

10. The device of claim 9, wherein the proximal end is configured for screwdriver or wrench engagement and the distal end is tapered.

11. The device of claim 1, wherein the fulcrum comprises a plurality of fulcrums spaced apart from each other along the elongated member.

12. The device of claim 1, wherein the fulcrum comprises a pulley, a pin, or a ledge defined in the slot.

13. The device of claim 1, wherein the slot comprises a first slot and a second slot generally opposite the elongated member from the first slot.

14. The device of claim 13, wherein the first slot extends distally further along the elongated member than the second slot.

15. The device of claim 1, wherein the slot comprises a first fulcrum ledge and a second fulcrum ledge, the first fulcrum ledge being located more distal on the elongated member than the second fulcrum ledge.

16. The device of claim 15, wherein the first fulcrum ledge is located on generally an opposite side of the elongated member from the second fulcrum ledge.

17. The device of claim 15, wherein the slot further comprises a segment routed both distal-proximal along the elongated member and circumferentially about at least a portion of a circumference of the elongated member.

18. The device of claim 1, further comprising a radiopaque marker indicating a location of the fulcrum.

19. A device for cutting bone in performing a pedicle subtraction osteotomy, the device comprising:
an elongated member comprising a fulcrum and a length distally terminating in a distal end, the elongated member configured to anchor in bone when the length is imbedded in bone, and the fulcrum located proximal the distal end at a location of the elongated member that would be within bone when the length is imbedded in bone; and
a flexible cutting member supported on the elongated member such that the flexible cutting member can be displaced back and forth about the fulcrum in a sawing fashion.

20. The device of claim 19, wherein the elongated member is configured such that a first portion of the flexible cutting member on a first side of the fulcrum extends through the elongated member to exit the elongated member at a proximal end of the elongated member.

21. The device of claim 20, wherein the elongated member is further configured such that a second portion of the flexible cutting member on a second side of the fulcrum opposite the first side of the fulcrum exits the elongated member through a slot extending longitudinally along a side of the elongated member.

22. The device of claim 21, wherein the slot extends proximal-distal generally parallel to a longitudinal axis of the elongated member.

23. The device of claim 21, wherein the proximal end comprises a head configured for screwdriver or wrench engagement.

24. The device of claim 23, wherein the slot extends through a side of the head.

25. The device of claim 21, wherein the elongated member further comprises a channel extending along a longitudinal axis of the elongated member, the slot opening into the channel and the first portion of the flexible cutting member extending through the channel in extending through the elongated member.

26. The device of claim 21, wherein the fulcrum comprises a pulley, a pin, or a ledge defined in the slot.

27. The device of claim 19, wherein, in being configured to anchor in bone, the elongated member comprises a threaded exterior configuration.

28. The device of claim 19, wherein the flexible cutting member comprises an abrasive or tooth equipped wire, cable or rope.

29. The device of claim 19, further comprising a radiopaque marker indicating a location of the fulcrum.

30. A method of cutting bone in performing a pedicle subtraction osteotomy, the method comprising:
anchoring a first elongated member to a first location on the bone such that a fulcrum supported on the first elongated member is located inside the bone; and
moving a flexible cutting member back and forth about the fulcrum to cause the flexible cutting member to cut the bone.

31. The method of claim 30, wherein the fulcrum is supported on the first elongated member near a distal end of the first elongated member, the distal end of the first elongated member being embedded inside the bone.

32. The method of claim 30, further comprising causing at least a portion of the flexible cutting member to extend from an elongated slot defined in the first elongated member.

33. The method of claim 30, wherein anchoring comprises screwing the first elongated member into the bone.

34. The method of claim 30, wherein anchoring comprises screwing the first elongated member into the bone such that at least a distal end of the elongated member becomes embedded in the bone.

35. The method of claim 30, wherein the first location on the bone comprises a first vertebral pedicle subsequent to a laminectomy and facetectomy.

36. The method of claim 30, further comprising:
anchoring a second elongated member to a second location on the bone such that a fulcrum supported on the second elongated member is located inside the bone; and
causing the flexible member to extend across the fulcrum of the first elongated member and the fulcrum of the second elongated member,
wherein moving the flexible cutting member back and forth about the fulcrum to cause the flexible cutting member to cut the bone further comprises moving the flexible cutting member back and forth about the first and second fulcrums to cause the flexible cutting member to cut the bone.

37. The method of claim 36, wherein the second location on the bone comprises a second vertebral pedicle subsequent to a laminectomy and facetectomy.

38. The method of claim 37, further comprising causing the flexible cutting member to extend across a vertebra between the first and second locations and under a dura.

39. The method of claim 38, locating a shield between the flexible cutting member and a nerve structure.

40. The method of claim 39, wherein the shield is located between the first and second locations.

41. The method of claim 36, further comprising joining a first segment of the flexible cutting member to a second segment of the flexible cutting member to form the flexible cutting member.

42. The method of claim 30, wherein the elongated member comprises a threaded exterior configuration.

43. The method of claim 30, wherein the flexible cutting member comprises an abrasive or tooth equipped wire, cable or rope.
